SEQUENCE LISTING (8 sequences)

SEQ ID NO: 1 - DNA, 12 bp, synthetic construct
  source 1..12: mol_type = other DNA; note = polynucleotide
  modified_base 5: mod_base = OTHER; note = hmC (5-hydroxymethylcytosine)
  modified_base 12: mod_base = OTHER; note = mC (5-methylcytosine)
ccgtcggaccgc

SEQ ID NO: 2 - DNA, 12 bp, synthetic construct
  source 1..12: mol_type = other DNA; note = polynucleotide
  modified_base 5: mod_base = OTHER; note = caC (5-carboxylcytosine)
  modified_base 12: mod_base = OTHER; note = caC (5-carboxylcytosine)
ccgtcggaccgc

SEQ ID NO: 3 - DNA, 12 bp, synthetic construct
  source 1..12: mol_type = other DNA; note = polynucleotide
  modified_base 5: mod_base = OTHER; note = caC* (caC* is illustrated at operation 230 in FIG. 2 includes moiety X corresponding to the nucleophilic molecule as reacted with the activated 5-carboxyl)
  modified_base 12: mod_base = OTHER; note = caC* (CaC* is illustrated at operation 230 in FIG. 2 includes moiety X corresponding to the nucleophilic molecule as reacted with the activated 5-carboxyl)
ccgtcggaccgc

SEQ ID NO: 4 - DNA, 12 bp, synthetic construct
  source 1..12: mol_type = other DNA; note = polynucleotide
  modified_base 5: mod_base = OTHER; note = T* (T* is illustrated at operation 240 of FIG. 2, includes X* which represents the further reacted nucleophilic molecule and T* refers to the product having a pattern of electron density which is sufficiently similar to that of thymine (T) to be amplified as T during polymerase chain reaction (PCR))
  modified_base 12: mod_base = OTHER; note = T* (T* is illustrated at operation 240 of FIG. 2, includes X* which represents the further reacted nucleophilic molecule and T* refers to the product having a pattern of electron density which is sufficiently similar to that of thymine (T) to be amplified as T during polymerase chain reaction (PCR))
ccgttggaccgt

SEQ ID NO: 5 - DNA, 12 bp, synthetic construct
  source 1..12: mol_type = other DNA; note = polynucleotide
ccgttggaccgt

SEQ ID NO: 6 - DNA, 12 bp, synthetic construct
  source 1..12: mol_type = other DNA; note = complementary sequence
acggtccaacgg

SEQ ID NO: 7 - DNA, 12 bp, synthetic construct
  source 1..12: mol_type = other DNA; note = polynucleotide
ccgtcggaccgc

SEQ ID NO: 8 - DNA, 12 bp, synthetic construct
  source 1..12: mol_type = other DNA; note = complementary sequence
gcggtccgacgg

(19) United States
(12) Patent Application Publication
(10) Pub. No.: US 2025/0320549 A1
(43) Pub. Date: Oct. 16, 2025

(54) Methods of modifying methylcytosine or derivative thereof using a nucleophilic molecule, and methods of using the same to detect the methylcytosine or derivative thereof in a polynucleotide

(71) Applicant: ILLUMINA, INC., San Diego, CA (US)

(72) Inventors: Elena Cressina, Cambridge (GB); Carole Anastasi, Cambridge (GB); Mykhailo Vybornyi, Cambridge (GB)

(21) Appl. No.: 18/873,629

(22) PCT Filed: Aug. 25, 2023
(86) PCT No.: PCT/EP2023/073380

§ 371 Date: Dec. 10, 2024

Related U.S. Application Data
(60) Provisional application No. 63/401,020, filed on Aug. 25, 2022.

(51) Int. Cl. C12Q 1/6869; C07H 21/04; C12N 9/02; C12P 19/34; C12Q 1/6806

(52) U.S. Cl. CPC C12Q 1/6869; C07H 21/04; C12N 9/0071; C12P 19/34; C12Q 1/6806; C12Y 114/11

(57) ABSTRACT

Disclosed herein are methods of modifying 5-methylcytosine (5-mC), 5-hydroxymethylcytosine (5-hmC), or 5-formlcytosine (5-fC) in a polynucleotide. The method may include oxidizing the 5-mC, 5-hmC, or 5-fC to 5-carboxylcytosine (5-caC); activating the 5-carboxyl group of the 5-caC; and reacting the activated 5-carboxyl group with a nucleophilic molecule to form a product. In some examples, the product may be used to detect the 5-mC, 5-hmC, or 5-fC in the polynucleotide.

### CROSS-REFERENCE TO RELATED APPLICATION

This application claims the benefit of U.S. Provisional Patent Application No. 63/401,020, filed Aug. 25, 2022 and entitled “Methods of Modifying Methylcytosine or Derivative Thereof Using a Nucleophilic Molecule, and Methods of Using the Same to Detect the Methylcytosine or Derivative Thereof in a Polynucleotide,” the disclosure of which is hereby incorporated by reference in its entirety.

### FIELD

This application relates to modifying methylcytosine, and using the modified methylcytosine to detect the methylcytosine in a polynucleotide.

### STATEMENT REGARDING SEQUENCE LISTING

The Sequence Listing associated with this application is provided in xml format, and is hereby incorporated by reference into the specification. The name of the xml file containing the Sequence Listing is 85491_05516.xml. The xml file is 14.2 KB, was created on Aug. 15, 2023, and is being submitted electronically via EFS-web.

### BACKGROUND

Within living organisms, such as humans, selected cytosines in the genome may become methylated. A common method used to detect methylated cytosines is sodium bisulfite sequencing. One issue with this method is that it often results in greater than 95% of the input DNA being degraded. Borane-containing compounds can be used in various protocols to detect methylated cytosines. However, previously known boranes can also degrade DNA. Thus, new methods and compositions are needed to detect methylated DNA that reduces DNA degradation.

### SUMMARY

Examples provided herein are related to methods of modifying methylcytosine or a derivative thereof using a nucleophilic molecule, and methods of using the same to detect the methylcytosine or derivative thereof in a polynucleotide.

Some examples herein provide a method of modifying 5-methylcytosine (5-mC), 5-hydroxymethylcytosine (5-hmC), or 5-formlcytosine (5-fC) in a polynucleotide. The method may include oxidizing the 5-mC, 5-hmC, or 5-fC to 5-carboxylcytosine (5-caC). The method may include activating the 5-carboxyl group of the 5-caC. The method may include reacting the activated 5-carboxyl group with a nucleophilic molecule to form a product.

In some examples, a ten-eleven translocation (TET) dioxygenase is used to oxidize the 5-mC, 5-hmC, or 5-fC to 5-caC. In some examples, oxidizing 5-fC to 5-carboxylcytosine (5-caC) includes contacting the 5-mC, 5-hmC, or 5-fC with one or more chemical reagents.

In some examples, the 5-carboxyl group of the 5-caC is activated using 4-(4,6-dimethoxy-1,3,5-triazin-2-yl)-4-methyl-morpholinium chloride (DMTMM), 1-ethyl-3-(3′-(dimethylamino)propyl)carbodiimide (EDC), EDC in combination with N-hydroxylsuccinimide (NHS), ethyl 2-cyano-2-(hydroxylamino)acetate uronium salt (COMU), N,N′-carbonyldiimidazole (CDI), or O-(1,2-dihydro-2-oxo-1-pyridyl-N,N,N′,N′-tetramethyluronium tetrafluoroborate (TPTU).

In some examples, the nucleophilic molecule includes a first moiety, a methylene group, and a second moiety coupled to the first moiety via the methylene group, and the reacting includes the methylene group attacking the activated 5-carboxyl group.

In some examples, the first and second moieties include respective electron-withdrawing groups. In some examples, the nucleophilic molecule is selected from the group consisting of:

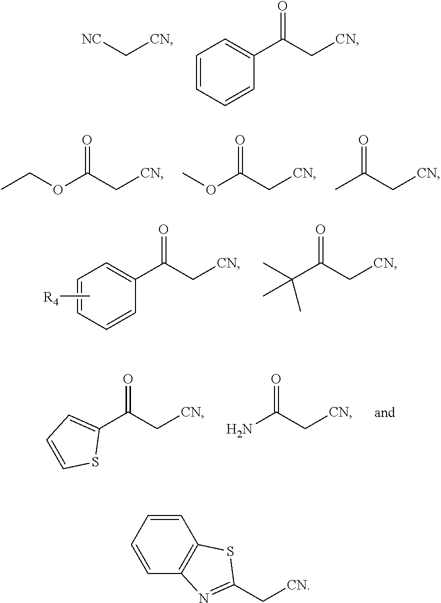

In some examples, the first moiety includes a cyano moiety. In some examples, the second moiety includes a cyano moiety. In some examples, R₄ is alkyl, alkenyl, alkynyl, alkoxy, alkylamino, cyano, nitro, or halo.

In some examples, the nucleophilic molecule is selected from the group consisting of:

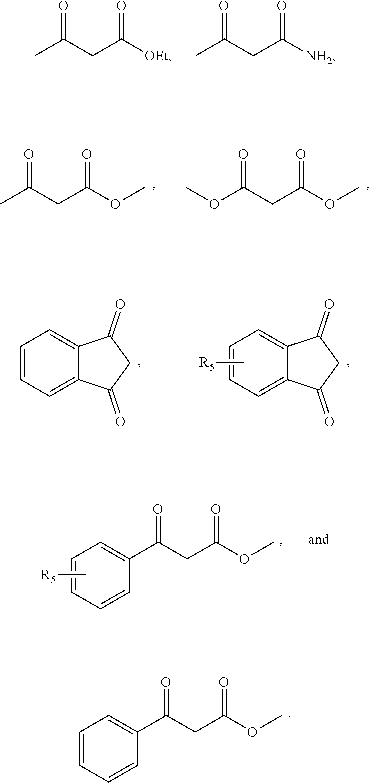

In some examples, R₅ or R₆ is an electron-withdrawing group. In some examples, the electron-withdrawing group is cyano, carboxy, or halo.

In some examples, the exocyclic amine of the 5-caC participates in the product rearranging.

In some examples, the product is cyclic. In some examples, the product includes:

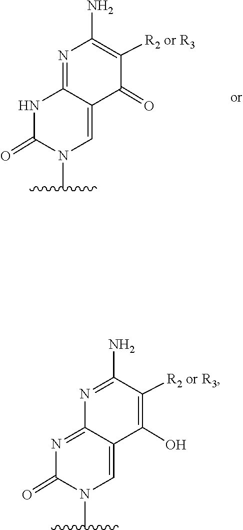

wherein R₂ or R₃ includes an electron withdrawing group.

Some examples herein provide a method of detecting 5-methylcytosine (5-mC), 5-hydroxymethylcytosine (5-hmC), or 5-formylcytosine (5-fC) in a polynucleotide. The method may include modifying the 5-mC, 5-hmC, or 5-fC using the method of any of the above examples to generate a modified polynucleotide including the product. The method may include detecting the 5-mC, 5-hmC, or 5-fC using the modified polynucleotide.

In some examples, the detecting includes generating a first amplicon of the modified polynucleotide, the first amplicon including adenine (A) at a location complementary to the product. In some examples, the detecting includes generating a second amplicon of the first amplicon, the second amplicon including thymine (T) at a location complementary to the A.

In some examples, the detecting includes sequencing the first amplicon, the second amplicon, or both the first amplicon and the second amplicon. In some examples, the detecting includes identifying the 5-mC or 5-hmC based on the first A in the first amplicon, the first T in the second amplicon, or both the first A in the first amplicon and the first T in the second amplicon.

Some examples herein provide an isolated polynucleotide from an extracellular fluid sample. The polynucleotide may include a product of a reaction between 5-carboxylcytosine (5-caC) and a nucleophilic molecule including a methylene group and first and second electron-withdrawing groups.

In some examples, the nucleophilic molecule is selected from the group consisting of:

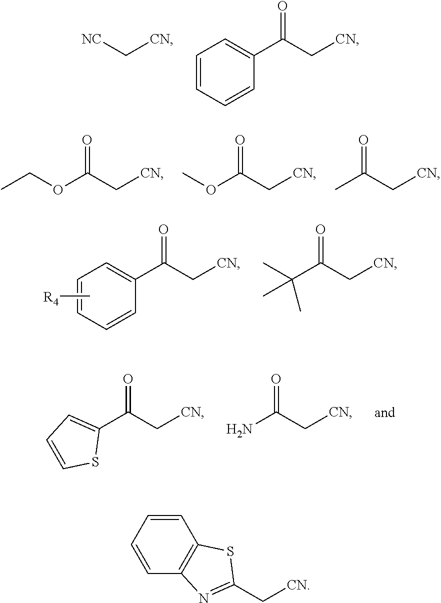

In some examples, the first moiety includes a cyano moiety. In some examples, the second moiety includes a cyano moiety. In some examples, R₄ is alkyl, alkenyl, alkynyl, alkoxy, alkylamino, cyano, nitro, or halo.

In some examples, the nucleophilic molecule is selected from the group consisting of:

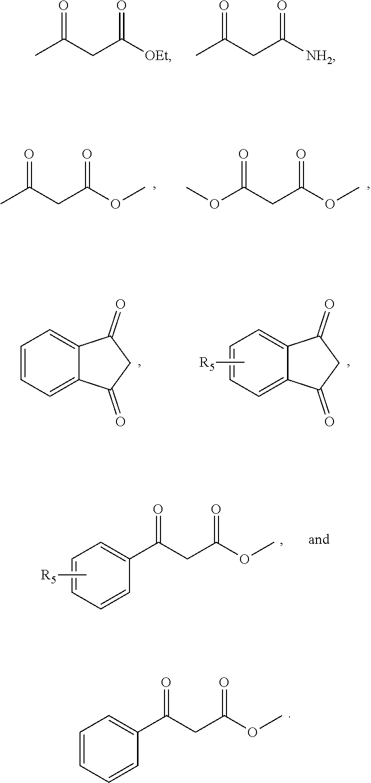

In some examples, R₅ or R₆ is an electron-withdrawing group. In some examples, the electron-withdrawing group is cyano, carboxy, or halo.

In some examples, the exocyclic amine of the 5-caC participates in the product rearranging.

In some examples, the product is cyclic. In some examples, the product includes:

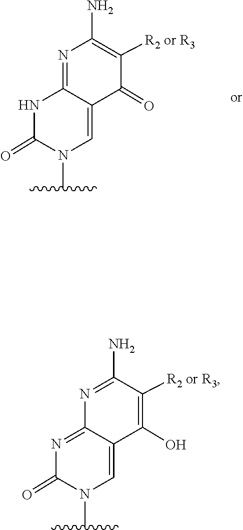

wherein R₂ or R₃ includes an electron withdrawing group.

Some examples herein provide a double-stranded polynucleotide. The double-stranded polynucleotide may include the polynucleotide of any of the above examples; and a second polynucleotide hybridized to the polynucleotide and including adenine (A) at a location complementary to the product.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1 schematically illustrates an example flow of operations in a method for modifying methylcytosine or derivative thereof using a nucleophilic molecule.

FIG. 2 schematically illustrates example structures formed using operations described with reference to FIG. 1.

FIG. 3A schematically illustrates example hydrogen bonding between carboxylcytosine and guanine in a double-stranded polynucleotide.

FIG. 3B schematically illustrates example hydrogen bonding between carboxylcytosine, modified using a nucleophilic molecule in a manner such as provided herein, and adenine in a double-stranded polynucleotide.

FIG. 4 schematically illustrates example operations for detecting methylcytosine or derivative thereof in a polynucleotide using operations, structures, and hydrogen bonding such as described with reference to FIGS. 1, 2, and 3A-3B.

FIGS. 5A-5C illustrate ultra performance liquid chromatography (UPLC) traces at 280 nm of example reaction mixtures.

FIG. 5D illustrates the absorption profile of example reaction products.

FIG. 5E illustrates mass spectrometry profiles of example reaction products.

### DETAILED DESCRIPTION

Examples provided herein are related to methods of modifying methylcytosine or a derivative thereof using a nucleophilic molecule, and methods of using the same to detect the methylcytosine or derivative thereof in a polynucleotide.

For example, as provided herein, methylcytosine (mC), or a derivative thereof such as hydroxymethylcytosine (hmC) or formylcytosine (fC), in a polynucleotide may be detected using a workflow in which the mC, hmC, or fC is enzymatically or chemically oxidized to carboxylcytosine (caC), and a nucleophilic molecule is used to modify the caC to generate a product. The polynucleotide then may be amplified using polymerase chain reaction (PCR), during which the modified caC is amplified as thymine (T) and as such the mC, hmC, or fC is sequenced as T. In comparison, the unmethylated C is amplified, and sequenced, as C. Thus, any Cs in the sequence may be identified as corresponding to C because they had not been converted to T, while any mC, hmC, or fC in the sequence may be identified as corresponding to mC, hmC, or fC because they had been converted to T. Such a scheme may be referred to as a “four-base” sequencing scheme because any unmethylated C is sequenced as C, providing the ability to obtain both sequence and methylation information from the processed polynucleotide. As provided herein, the present methods use reagents that are sufficiently water-soluble and mild as to be used with polynucleotides in a practical commercial implementation, and substantially without damaging the polynucleotides thus improving yield and accuracy of detecting mC, hmC, or fC while preserving the polynucleotide sequence itself as well.

First, some terms used herein will be briefly explained. Then, some example methods for modifying mC or its derivatives, structures formed using such methods, and methods for detecting mC or its derivatives in a polynucleotide using the present subject matter, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes disassociate from one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 5-halouracil, 5-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “methylcytosine” or “mC” refers to cytosine that includes a methyl group (—CH₃ or -Me). The methyl group may be located at the 5 position of the cytosine, in which case the mC may be referred to as 5-mC.

As used herein, a “derivative” of methylcytosine refers to methylcytosine having an oxidized methyl group. A nonlimiting example of an oxidized methyl group is hydroxymethyl (—CH₂OH), in which case the mC derivative may be referred to as hydroxymethylcytosine or hmC. Another nonlimiting example of an oxidized methyl group is formyl group (—CHO) in which case the mC derivative may be referred to as formylcytosine or fC. Another nonlimiting example of an oxidized methyl group is carboxyl (—COOH), in which case the mC derivative may be referred to as carboxylcytosine or caC. The oxidized methyl group may be located at the 5 position of the cytosine, in which case the hmC may be referred to as 5-hmC, the fC may be referred to as 5-fC, or the caC may be referred to as 5-caC. The fC optionally may be present in an acetal form (—CH(OH)₂). The caC optionally may be present in a salt form (—COO—).

As used herein, the terms “electron donating group,” “electron-donor,” and the like are intended to refer to a group that releases electron density from itself to adjacent atoms, thereby increasing the electron density of the adjacent atoms.

As used herein, the terms “electron withdrawing group,” “electron-acceptor,” and the like are intended to refer to a group that draws electron density from adjacent atoms to itself, thereby reducing electron density of the adjacent atoms.

As used herein, the term “aqueous solution” is intended to refer to any solution in which water functions as a solvent.

As used therein, the term “activating a carboxyl group” is intended to refer to reacting the —OH group of the carboxyl group with any suitable chemical and/or enzymatic reagents that make it easier to replace the —OH group of the carboxylic acid with a nucleophilic molecule.

### Methods of Modifying Methylcytosine or a Derivative Thereof Using a Nucleophilic Molecule

Some examples provided herein relate to modifying methylcytosine (5-mC) or a derivative thereof (e.g., 5-hmC or 5-fC) using a nucleophilic molecule.

More specifically, the present inventors have recognized that 5-mC, 5-hmC, or f-5C in a polynucleotide may be converted to caC, and the caC selectively chemically reacted with a nucleophilic molecule to form a product.

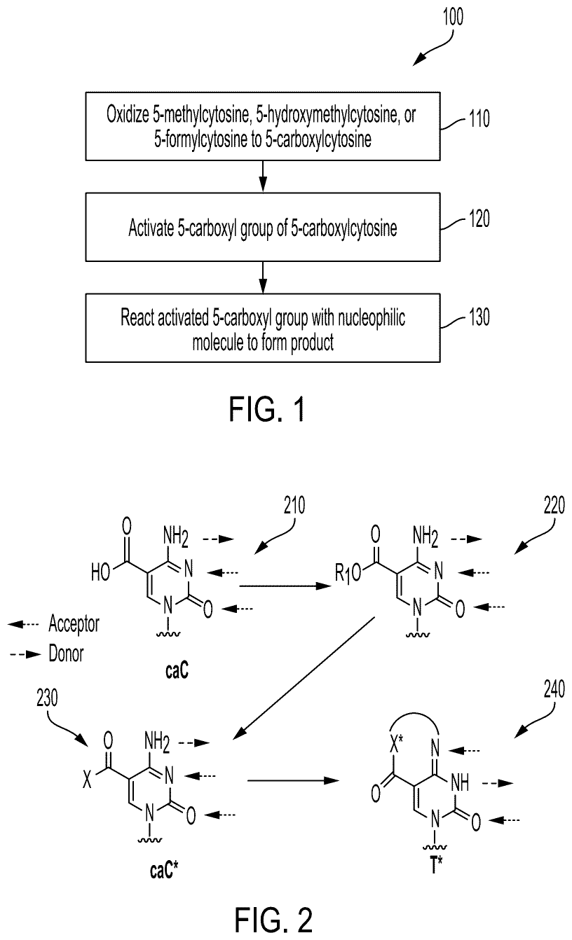

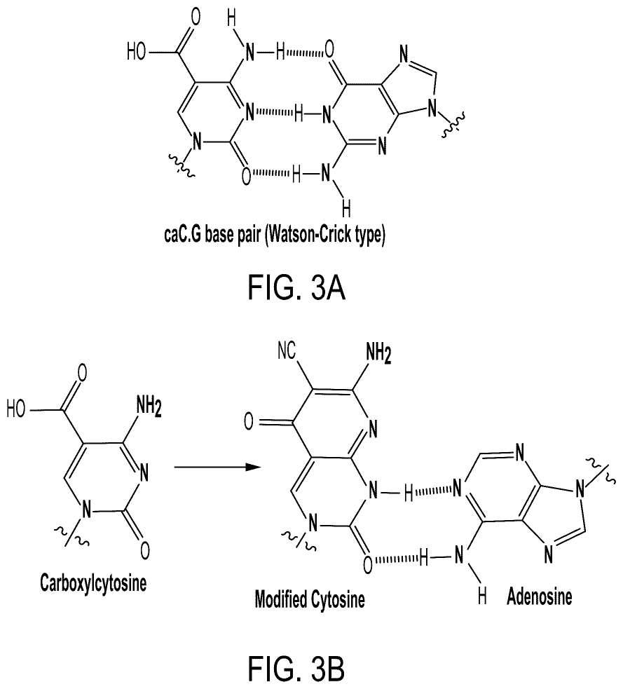

FIG. 1 schematically illustrates an example flow of operations in a method for modifying methylcytosine or derivative thereof using a nucleophilic molecule, and FIG. 2 schematically illustrates example structures formed using operations described with reference to FIG. 1. Referring first to FIG. 1, method 100 may include oxidizing 5-mC, 5-hmC, or 5-fC to 5-caC (operation 110). The oxidation may be performed using any suitable combination of chemical and/or enzymatic reagents. In some examples using an enzymatic reagent, a ten-eleven translocation (TET) dioxygenase is used to oxidize the 5-mC, 5-hmC, or 5-fC to 5-caC. In some nonlimiting examples using one or more chemical reagents, 5-mC may be oxidized to 5-caC using menadione, ultraviolet (UV) radiation at 365 nm, under oxygen, followed by 2,2,6,6-tetramethyl-1-piperidinyloxy free radical (TEMPO)/bis(acetoxyiodobenzene) (BAIB) in a manner such as described in Kore et al., “Concise synthesis of 5-methyl, 5-formyl, and 5-carboxy analogues of 2′-deoxycytidine-5′-triphosphate,” Tetrahedron letters 54(39): 5325-5327 (2013), the entire contents of which are incorporated by reference herein. In other nonlimiting examples using one or more chemical reagents, 5-hmC or 5-fC may be oxidized to 5-caC using TEMPO/BAIB in a manner such as described in Sun et al., “Efficient synthesis of 5-hydroxymethyl-, 5-formyl-, and 5-carboxyl-2′-deoxycytidine and their triphosphates,” RSC Advances 4(68): 36036-36039 (2014), the entire contents of which are incorporated by reference herein. In still other nonlimiting examples using one or more chemical reagents, an iron(IV)-oxo complex is used to oxidize 5-mC to 5-caC in a manner such as described in Schmidl et al., “Biomimetic iron complex achieves TET enzyme reactivity,” Angewandte Chemie Int′l Ed. 60(39): 21457-21463 (2021), the entire contents of which are incorporated by reference herein.

The 5-mC, 5-hmC, or 5-fC may be present in a polynucleotide, and the polynucleotide may be present in an extracellular sample. As such, the 5-caC resulting from operation 110 may be present in the polynucleotide. An example structure of caC, which may be present in a polynucleotide and resulting from operation 110, is illustrated at operation 210 in FIG. 2.

Referring again to FIG. 1, method 100 may include activating the 5-carboxyl group of the 5-caC (operation 120). Such activation may be performed using any suitable combination of chemical and/or enzymatic reagents. In some examples, the 5-carboxyl group of the 5-caC is activated using 4-(4,6-dimethoxy-1,3,5-triazin-2-yl)-4-methyl-morpholinium chloride (DMTMM), 1-ethyl-3-(3′-(dimethylamino)propyl)carbodiimide (EDC), EDC in combination with N-hydroxylsuccinimide (NHS), ethyl 2-cyano-2-(hydroxylamino)acetate uronium salt (COMU), N,N′-carbonyldiimidazole (CDI), or O-(1,2-dihydro-2-oxo-1-pyridyl-N,N,N′,N′-tetramethyluronium tetrafluoroborate (TPTU). Nonlimiting examples of such reagents are illustrated below:

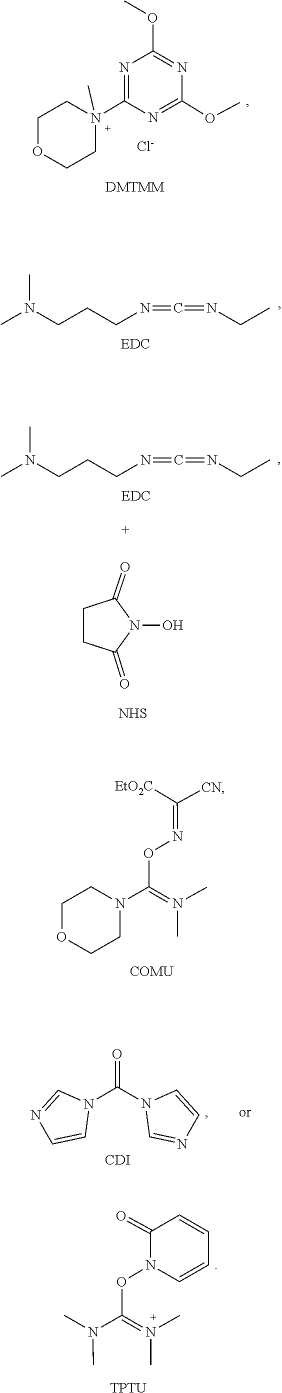

For further details regarding reagents for activating carboxyl groups, see the following references, the entire contents of which are incorporated by reference herein: Badland et al., “A comparative study of amide-bond forming reagents in aqueous media—Substrate scope and reagent compatibility,” Tetrahedron Letters 46(15): 4391-4394 (2017); and El-Faham et al., “COMU: A safer and more effective replacement for benzotriazole-uronium coupling reagents,” Chem. Eur. J. 15(37): 9404-9416 (2009). An example structure of an activated ester, which may be present in a polynucleotide and resulting from operation 120, is illustrated at operation 220 in FIG. 2. In the activated ester illustrated at operation 220, R₁ may include the product of reaction between the —OH of the caC's carboxyl group and any of the reagents illustrated above or any other suitable chemical or enzymatic reagent.

Referring again to FIG. 1, method 100 may include reacting the activated 5-carboxyl group with a nucleophilic molecule to form a product (operation 130). An example structure of a product resulting from operation 130, which product may be present in a polynucleotide, is illustrated at operation 230 in FIG. 2. The product “caC*” illustrated at operation 230 in FIG. 2 includes moiety X corresponding to the nucleophilic molecule as reacted with the activated 5-carboxyl.

In some examples, the nucleophilic molecule with which the activated 5-carboxyl group is reacted during operation 130 includes a first moiety (R₂), a methylene group, and a second moiety (R₃) coupled to the first moiety via the methylene group, and wherein the reacting includes the methylene group attacking the activated 5-carboxyl group. The first and second moieties may include respective electron-withdrawing groups. In some examples, the first moiety (R₂) includes a cyano (—CN) moiety. An example scheme for reaction between such a nucleophilic molecule and the activated ester resulting from operation 110 is illustrated below:

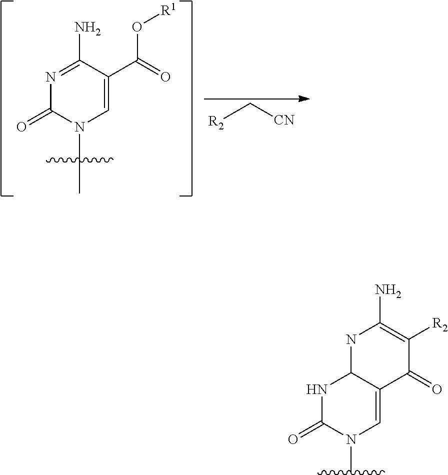

Nonlimiting examples of nucleophilic molecules R₂∧CN that may be used in such a scheme may be selected from the group consisting of:

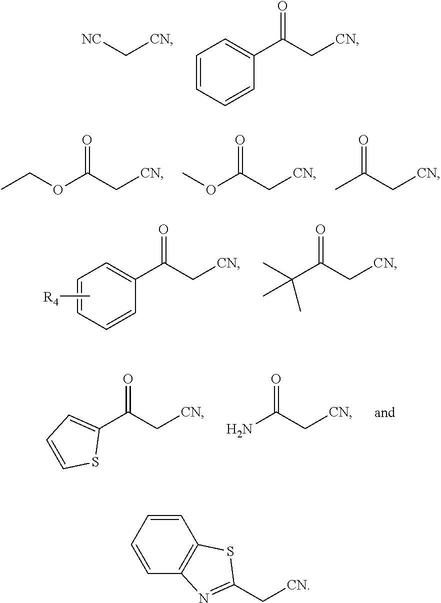

In one nonlimiting example, both the first moiety (R₂) and second moiety (R₃) include a cyano moiety. Illustratively, both the first moiety (R₂) and second moiety (R₃) may consist essentially of a cyano moiety; that is, the nucleophilic molecule may be malononitrile (NC∨CN). In some examples R₄ may include alkyl, alkenyl, alkynyl, alkoxy, alkylamino, cyano, nitro, or halo.

It will be appreciated that other nucleophilic molecules may not necessarily include a cyano moiety. For example, the nucleophilic molecule may include a ketone. An example scheme for reaction between such a nucleophilic molecule and the activated ester resulting from operation 110 is illustrated below:

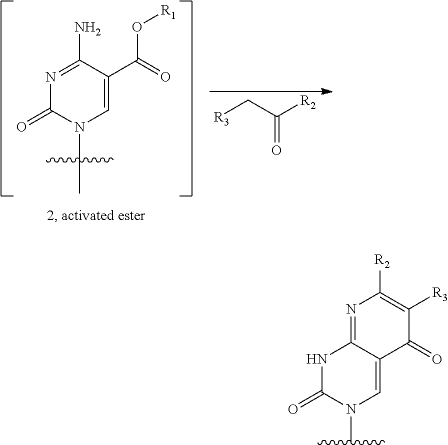

Nonlimiting examples of nucleophilic molecules

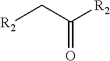

that may be used in such a scheme may be selected from the group consisting of:

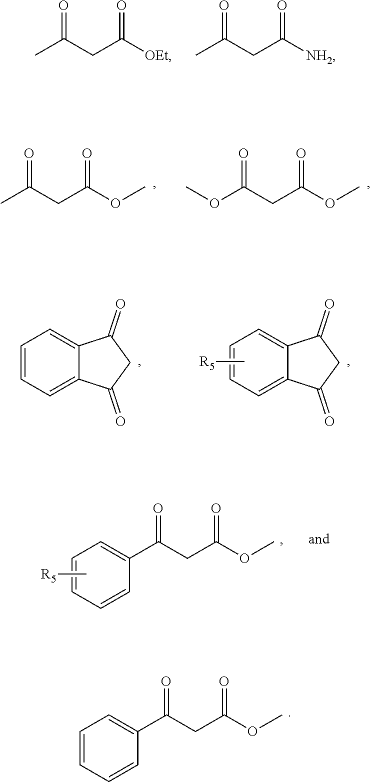

In some examples R₅ or R₆ may include an electron-withdrawing group, such as cyano, carboxy, or halo.

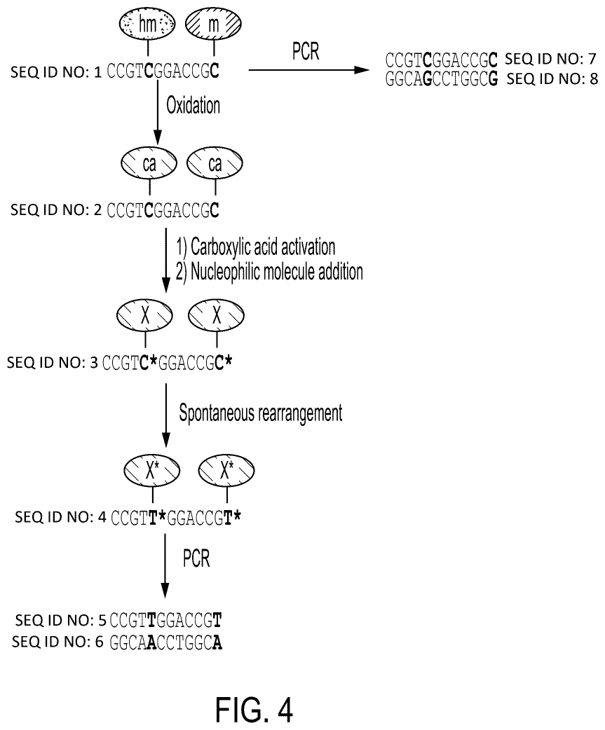

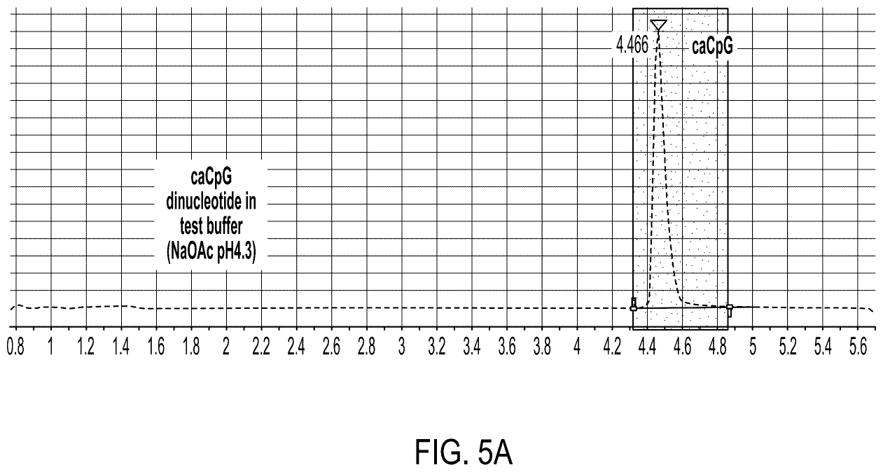

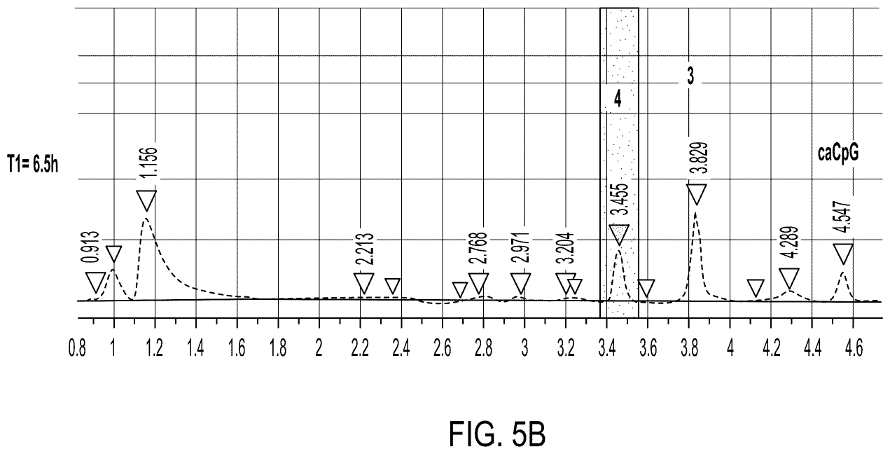

In a manner such as illustrated at operation 240 of FIG. 2, the product of reaction between the activated 5-carboxyl group of 5-caC and the nucleophilic molecule may rearrange. The product of such rearrangement optionally may be cyclic. Illustratively, the exocyclic amine of the 5-caC participates in the product rearranging. In cyclic product T* illustrated at operation 240 of FIG. 2, X* represents the further reacted nucleophilic molecule and T* refers to the product having a pattern of electron density which is sufficiently similar to that of thymine (T) to be amplified as T during polymerase chain reaction (PCR) in a manner such as will be described in greater detail below with reference to FIGS. 3 and 4. The cyclic product of such rearrangement may be able to tautomerize, e.g., between enol and ketone forms such as illustrated below:

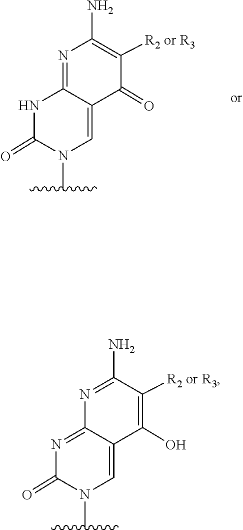

wherein R₂ or R₃ includes an electron withdrawing group.

Note that the operations illustrated in FIGS. 1 and 2 may be performed in any suitable order. For example, although FIG. 1 may appear to suggest that operations 120 and 130 are performed as separate operations, such operations may be performed concurrently, e.g., by contacting the 5-caC with both a carboxylic acid-activating reagent and the nucleophilic molecule.

### Methods of Detecting Methylcytosine or Derivative Thereof in a Polynucleotide

In some examples, operations and structures such as described with reference to FIGS. 1-2 are used to detect methylation of DNA, including detection of 5-mC, 5-hmC, and/or 5-fC.

The TAPS (TET-assisted pyridine borane sequencing) workflow is described in the following references, the entire contents of each of which are incorporated by reference herein: Liu et al., “Bisulfite-free direct detection of 5-methylcytosine at base resolution,” Nature Biotechnology 37: 424-429 (2019); Liu et al., “Accurate targeted long-read DNA methylation and hydroxymethylation sequencing with TAPS,” Genome Biology 21: Article no. 54 (2020); Liu et al., “Subtraction-free and bisulfite-free specific sequencing of 5-methycytosine and its oxidized derivatives at base resolution,” Nature Communications 12: Article no. 618 (2021); and International Publication No. WO 2019/136413 to Song et al. Briefly, and as described in these references, the TAPS workflow uses a ten-eleven translocation (TET) dioxygenase to oxidize any 5-mC, 5-hmC, and/or 5-fC in a polynucleotide to 5-caC.

As provided herein, 5-caC (which may be prepared using an enzymatic reagent such as TET, or using one or more chemical reagents such as described elsewhere herein), is reacted with a nucleophilic molecule to obtain a product having a pattern of electron density which is sufficiently similar to that of thymine (T) to be amplified as T during polymerase chain reaction (PCR). The product is sequenced as a T to detect locations where 5-mC, 5-hmC, or 5-fC had been located.

The present inventors have recognized that the previously known TAPS workflow presents several challenges which may impede practical commercial implementation. For example, reduction of 5-carboxylcytosine using the pyridine borane complex requires a long incubation time (e.g., about 16 hours) at low pH, high temperature, and a high concentration of reagent (e.g., about 1 M) in order to be efficient. It is believed that these reaction conditions may cause considerable degradation of the DNA, reducing reaction yield and particularly degrading heavily methylated regions. Additionally, the pyridine borane complex is highly toxic and volatile, and requires the use of specialized equipment (such as a fume hood) which may not be compatible with automated sample preparation as may be desirable for use in a commercial implementation. The picoline borane complex also is believed not to be suitable for commercial implementation for similar reasons.

The present inventors have recognized that operations such as described with reference to FIGS. 1 and 2 suitably may be used to modify any 5-mC, 5-hmC, and/or 5-fC in a polynucleotide to a detectable product with a higher efficiency, and with less damage to the polynucleotide, than may be achieved using pyridine borane or picoline borane such as used in the TAPS workflow. The present reagents are sufficiently water-soluble, reactive, and non-volatile as to be useful at mild pH and without the need for a fume hood or extended reaction times. As such, the present operations may be readily implemented in a practical commercial setting, and are expected to provide higher reaction yield and improved accuracy in detecting methylcytosine and its derivatives.

Without wishing to be bound by any theory, it is believed that by suitably selecting a nucleophilic molecule with which the activated ester of 5-caC may react, the product of the reaction (including any subsequent rearrangements) may have a pattern of electron density which is sufficiently similar to that of T to be amplified as T during PCR. For example, FIG. 3A schematically illustrates example hydrogen bonding between 5-caC and guanine (G) in a double-stranded polynucleotide. In a manner such as illustrated in FIG. 3A, the exocyclic primary amine in position 4 of 5-caC acts as an hydrogen-bond donor by sharing its hydrogen with the keto group in position 6 of G, while the heterocyclic tertiary amine in position 3 and keto group in position 2 of 5-caC act as hydrogen-bond acceptors with which the secondary amine in position 1 and exocyclic primary amine in position 2 of G respectively share hydrogens. As illustrated in FIG. 2, activation of the 5-caC's carboxyl group (operation 220), followed by reaction with a nucleophilic molecule to obtain product caC* (operation 230) and subsequent rearrangement to obtain product T* (operation 240) may convert the exocyclic, hydrogen-bond-donating primary amine in position 4 of 5-caC into an hydrogen-bond-accepting tertiary amine, and may convert the heterocyclic tertiary amine in position 3 of 5-caC into an hydrogen-bond-donating secondary amine. FIG. 3B schematically illustrates example hydrogen bonding between T* (caC modified using a nucleophilic molecule in a manner such as provided herein), and adenine in a double-stranded polynucleotide. In a manner such as illustrated in FIG. 3B, the heterocyclic secondary amine of T* (modified cytosine) acts as an hydrogen-bond-donor that shares its hydrogen with the heterocyclic tertiary amine in position 1 of A, while the keto group of T* acts as an hydrogen-bond-acceptor with which the exocyclic primary amine in position 6 of A shares hydrogen. Accordingly, it may be understood that operation 240's conversion of hydrogen-bond-donating primary amine of 5-caC into the hydrogen-bond-accepting tertiary amine of T* yields a moiety that may not hydrogen bond with a corresponding moiety of G. Additionally, it may be understood that operation 240's conversion of the heterocyclic tertiary amine of 5-caC into the hydrogen-bond-donating secondary amine of T* yields a moiety that may share its hydrogen with the tertiary amine in position 1 of A. Such modifications facilitate T* preferentially binding to A, rather than with G as is the case of caC.

FIG. 4 schematically illustrates example operations for detecting methylcytosine or derivative thereof in a polynucleotide using operations, structures, and hydrogen bonding such as described with reference to FIGS. 1, 2, and 3A-3B. The workflow (method) illustrated in FIG. 4 includes oxidizing any 5-methylcytosine, 5-hydroxymethylcytosine, or 5-formylcytosine in the polynucleotide to 5-carboxylcytosine. Illustratively, in the nonlimiting example shown in FIG. 4, the polynucleotide has the sequence CCGThmCGGACCGmC (SEQ ID NO: 1), and TET dioxygenase or any suitable chemical reagent(s) is used to oxidize the hmC and mC to caC in a manner similar to that described in the above-cited references, yielding the sequence CCGTcaCGGACCGcaC (SEQ ID NO: 2). The polynucleotide then is contacted with a carboxylic activator and nucleophilic molecule, in a manner such as described further above with reference to FIGS. 1 and 2. The reaction product caC* is represented in FIG. 4 as including moiety X corresponding to the nucleophilic molecule as reacted with the activated 5-carboxyl (similarly as described with reference to operation 230 of FIG. 2). In the nonlimiting example illustrated in FIG. 4, the reaction product caC* in the sequence CCGTeaC*GGACCGeaC* (SEQ ID NO: 3) spontaneously rearranges to T*, yielding the sequence CCGTT*GGACCGT* (SEQ ID NO: 4). Note that although such spontaneous rearrangement is illustrated as an operation of FIG. 4 for clarity, it should be appreciated that such rearrangement may occur spontaneously with addition of the nucleophilic molecule, as opposed to as a separate operation. The reaction product T* is represented in FIG. 4 as including moiety X* corresponding to the further reacted nucleophilic molecule (similarly as described with reference to operation 240 of FIG. 2).

The mC, hmC, and/or fC then may be detected using the T*. For example, as illustrated in FIG. 4, a first set of PCR reactions then may be performed on the product of the nucleophilic molecule addition and rearrangement(s) to generate amplicons of such product. In such amplicons, the T* (resulting from oxidation and subsequent reactions and rearrangement(s) such as described with reference to FIGS. 1 and 2) is amplified as T, illustratively yielding the sequence 5′-CCGTTGGACCGT-3′ (SEQ ID NO: 5) (and complementary sequence 3′-GGCAACCTGGCA-5′ (SEQ ID NO: 6)). Additionally, a second set of PCR reactions may be performed on a separate aliquot of the unreacted polynucleotide. In such amplicons, the mC, hmC, and fC are amplified as C, illustratively yielding the sequence 5′-CCGTCGGACCGC-3′ (SEQ ID NO: 7) (and complementary sequence 3′-GGCAGCCTGGCG-5′) (SEQ ID NO: 8). The locations in the target polynucleotide at which mC, hmC, or fC were located and at which T* was generated using the present operations, may be determined by comparing the sequence of the amplicons from the first set of PCR reactions to the sequence of amplicons from the second set of PCR reactions. Bases that are T (or A) in the amplicons from the first set of PCR reactions and that are C (or G) in the amplicons from the second set of PCR reactions may be identified as corresponding to mC, hmC, or fC because they were converted to T* using the present operations.

### WORKING EXAMPLES

The following examples are intended to be purely illustrative, and not limiting of the present invention.

### Example 1

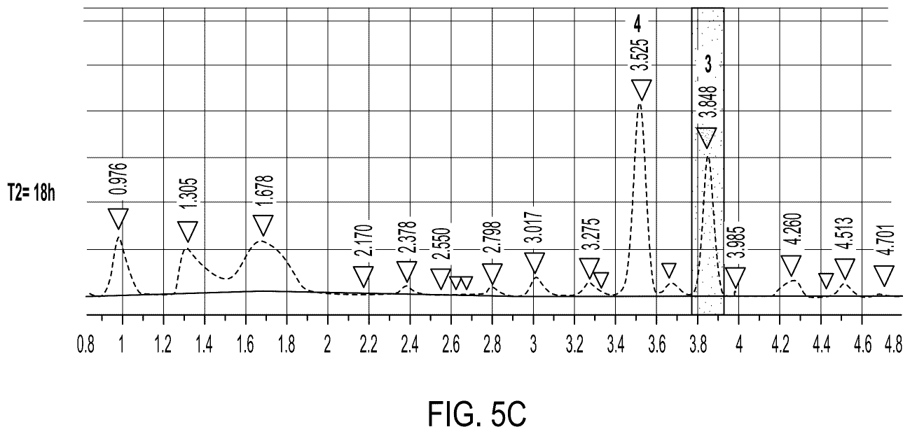

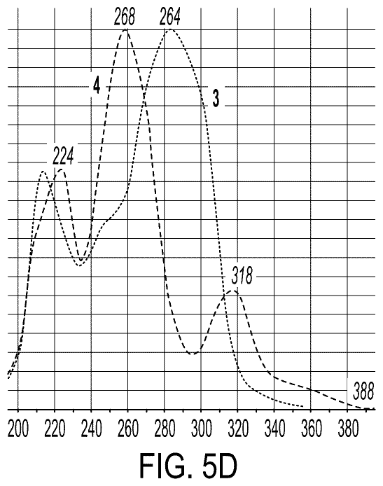

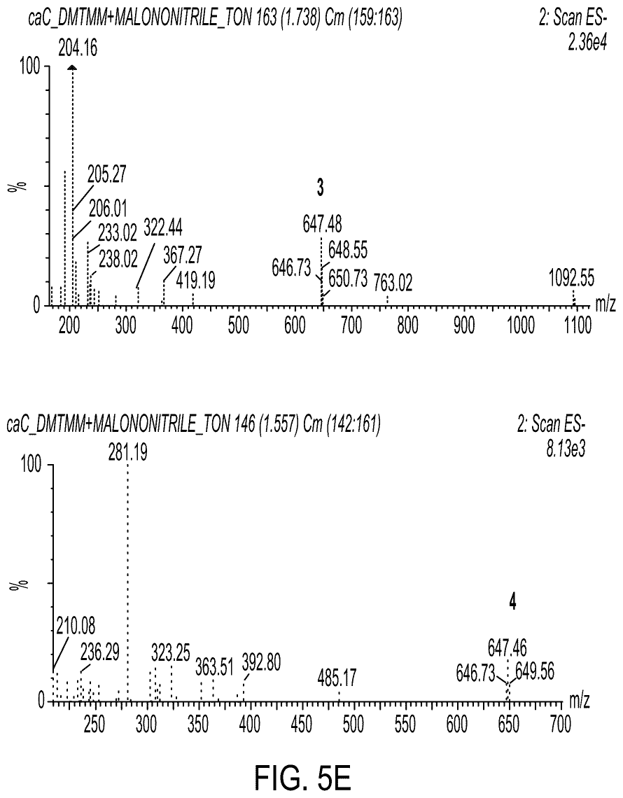

In a first example, caCpG (a dinucleotide including caC and G linked by a phosphate bond) at 0.5 mM was reacted with DMTMM at 250 mM and malononitrile at 150 mM in 300 mM aqueous MES buffer at a pH of about 5.0 and a temperature of about 40° C. for about 18 hours. The reaction progression and products formation were followed by taking aliquots of the reaction at defined time points (6.5 hours and 18 hours), and analyzing them by ultra performance liquid chromatography (UPLC) as shown in FIGS. 5A-5C and by liquid chromatography-mass spectrometry (LC-MS). By comparing the UPLC chromatogram of the starting material caCpG (UPLC peak retention time=4.5 min) shown in FIG. 5A with the chromatogram of the reaction at different time points FIGS. 5B-5C, it was observed that the reaction led to conversion of caC into two main products, cyclic caC-M (4) (UPLC peak retention time tr=3.5 min) and the open form (3) (UPLC peak retention time=3.8 min), both shown below:

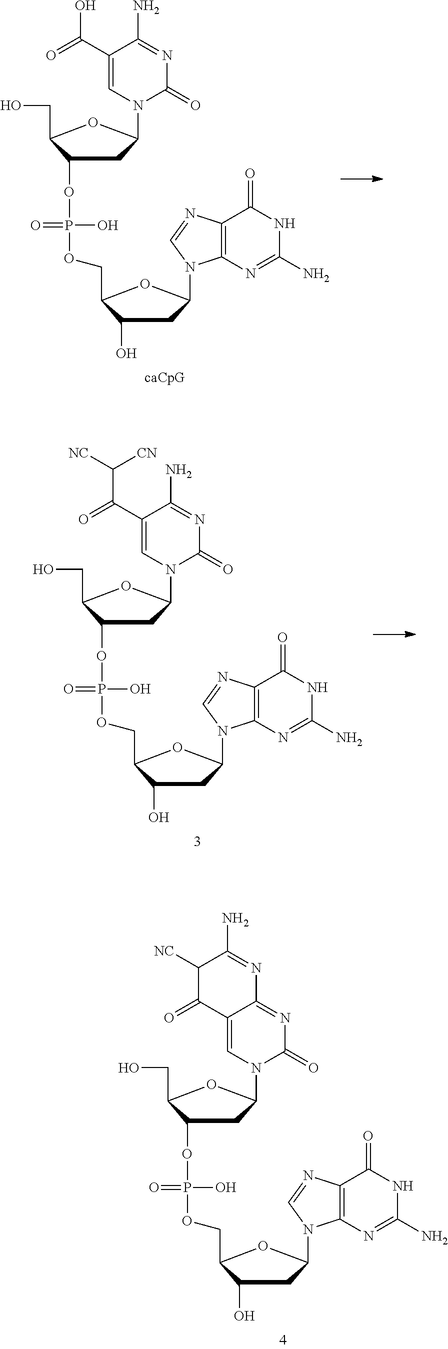 [SMILES: Nc1nc2c(ncn2[C@H]2C[C@@H](O)[C@@H](COP(=O)(O)O[C@@H]3C[C@H](n4cc(C(=O)O)c(N)nc4=O)O[C@@H]3CO)O2)c(=O)[nH]1 ; N#CC(C#N)C(=O)c1cn([C@H]2C[C@@H](OP(=O)(O)OC[C@H]3O[C@@H](n4cnc5c(=O)[nH]c(N)nc54)C[C@H]3O)[C@@H](CO)O2)c(=O)nc1N ; N#CC1C(=O)c2cn([C@H]3C[C@@H](OP(=O)(O)OC[C@H]4O[C@@H](n5cnc6c(=O)[nH]c(N)nc65)C[C@H]4O)[C@@H](CO)O3)c(=O)nc2N=C1N]

LC-MS analysis of the reaction aliquots indicated the formation of two products with relative m/z of 647 (negative mode) and m/z 649 (positive mode), which are consistent with the molecular weight of the structures 3 and 4. FIGS. 5A-5C illustrate ultra performance liquid chromatography (UPLC) traces at 280 nm of example reaction mixtures. More specifically, FIG. 5A illustrates the UPLC trace of starting material caCpG dinucleotide on its own in starting buffer. FIG. 5B illustrates the UPLC trace of caCpG+DMTMM+malononitrile after 6.5 h at 40C. FIG. 5C illustrates the UPLC trace of caCpG+DMTMM+malononitrile after 18 h at 40C. FIG. 5D illustrates the absorption profile of products (3) and (4). FIG. 5E illustrates mass spectrometry profiles (negative mode) of products (3) and (4). Upon longer incubation times, conversion of the open form (3) into the cyclic form (4) was observed. The cyclic form (4) was identified by its particular UV absorption spectrum, with a strong band at around 320 nm, as shown in FIG. 5D and mass spectrum shown in FIG. 5E. From this example, it may be understood that caC may be converted to a cyclic reaction product via carboxylic acid activation and reaction with a nucleophilic molecule.

### Example 2

5-mC, 5-hmC, and/or 5-fC in a polynucleotide fragment is converted to caC using TET dioxygenase in a manner such as described in Liu et al., “Bisulfite-free direct detection of 5-methylcytosine at base resolution,” Nature Biotechnology 37: 424-429 (2019). The caC is converted to the cyclic form (4) using reaction with DMTMM and malononitrile in the manner described with reference to Example 1. The resulting modified polynucleotide is amplified using PCR and sequenced. A second set of PCR reactions is performed on a separate aliquot of the unreacted polynucleotide. The sequence of the amplicons from the first set of PCR reactions is compared to the sequence of amplicons from the second set of PCR reactions. Bases that are T (or A) in the amplicons from the first set of PCR reactions and that are C (or G) in the amplicons from the second set of PCR reactions are identified as corresponding to mC, hmC, or fC.

### Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of modifying 5-methylcytosine (5-mC), 5-hydroxymethylcytosine (5-hmC), or 5-formlcytosine (5-fC) in a polynucleotide, the method comprising:
oxidizing the 5-mC, 5-hmC, or 5-fC to 5-carboxylcytosine (5-caC);
activating the 5-carboxyl group of the 5-caC; and
reacting the activated 5-carboxyl group with a nucleophilic molecule to form a product.

2. The method of claim 1, wherein a ten-eleven translocation (TET) dioxygenase is used to oxidize the 5-mC, 5-hmC, or 5-fC to 5-caC.

3. The method of claim 1, wherein oxidizing 5-fC to 5-carboxylcytosine (5-caC) comprises contacting the 5-mC, 5-hmC, or 5-fC with one or more chemical reagents.

4. The method of claim 1, wherein the 5-carboxyl group of the 5-caC is activated using 4-(4,6-dimethoxy-1,3,5-triazin-2-yl)-4-methyl-morpholinium chloride (DMTMM), 1-ethyl-3-(3′-(dimethylamino)propyl)carbodiimide (EDC), EDC in combination with N-hydroxylsuccinimide (NHS), ethyl 2-cyano-2-(hydroxylamino)acetate uronium salt (COMU), N,N′-carbonyldiimidazole (CDI), or O-(1,2-dihydro-2-oxo-1-pyridyl-N,N,N′,N′-tetramethyluronium tetrafluoroborate (TPTU).

5. The method of claim 1, wherein the nucleophilic molecule comprises a first moiety, a methylene group, and a second moiety coupled to the first moiety via the methylene group, and wherein the reacting comprises the methylene group attacking the activated 5-carboxyl group.

6. The method of claim 5, wherein the first and second moieties comprise respective electron-withdrawing groups.

7. The method of claim 6, wherein the nucleophilic molecule is selected from the group consisting of: 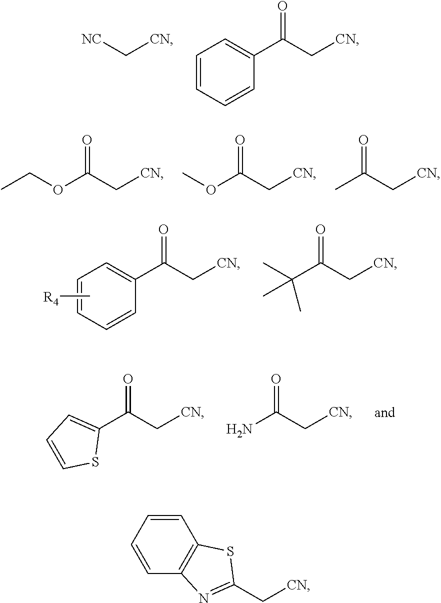
wherein R₄ is alkyl, alkenyl, alkynyl, alkoxy, alkylamino, cyano, nitro, or halo.

8. The method of claim 6, wherein the first moiety and/or the second moiety comprises a cyano moiety.

9. (canceled)

10. (canceled)

11. The method of claim 6, wherein the nucleophilic molecule is selected from the group consisting of: 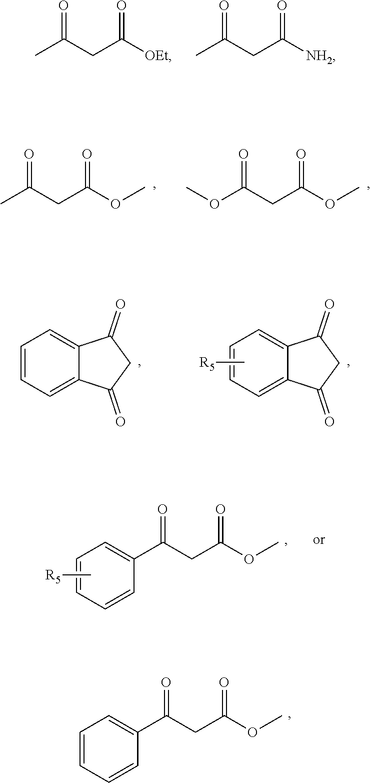
wherein R₅ or R₆ is cyano, carboxy, or halo.

12. (canceled)

13. (canceled)

14. The method of claim 1, wherein the exocyclic amine of the 5-caC participates in the product rearranging.

15. (canceled)

16. The method of claim 1, wherein the product comprises: 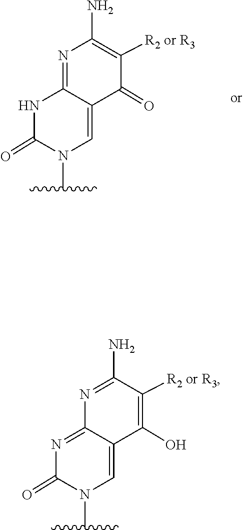
wherein R₂ or R₃ includes an electron withdrawing group.

17. A method of detecting 5-methylcytosine (5-mC), 5-hydroxymethylcytosine (5-hmC), or 5-formylcytosine (5-fC) in a polynucleotide, the method comprising:
modifying the 5-mC, 5-hmC, or 5-fC using the method of claim 1 to generate a modified polynucleotide comprising the product; and
detecting the 5-mC, 5-hmC, or 5-fC using the modified polynucleotide.

18. The method of claim 17, wherein the detecting comprises:
generating a first amplicon of the modified polynucleotide, the first amplicon including adenine (A) at a location complementary to the product;
generating a second amplicon of the first amplicon, the second amplicon including thymine (T) at a location complementary to the A;w
sequencing the first amplicon, the second amplicon, or both the first amplicon and the second amplicon; and
identifying the 5-mC or 5-hmC based on the first A in the first amplicon, the first T in the second amplicon, or both the first A in the first amplicon and the first T in the second amplicon.

19. An isolated polynucleotide from an extracellular fluid sample, the polynucleotide comprising a product of a reaction between 5-carboxylcytosine (5-caC) and a nucleophilic molecule comprising a methylene group and first and second electron-withdrawing groups.

20. The polynucleotide of claim 19, wherein the nucleophilic molecule is selected from the group consisting of: 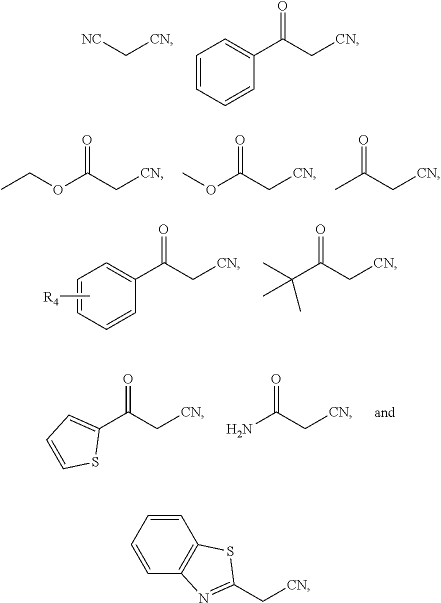
wherein R₄ is alkyl, alkenyl, alkynyl, alkoxy, alkylamino, cyano, nitro, or halo.

21. The polynucleotide of claim 19, wherein the first moiety and/or the second moiety comprises a cyano moiety.

22. (canceled)

23. (canceled)

24. The polynucleotide of claim 19, wherein the nucleophilic molecule is selected from the group consisting of: 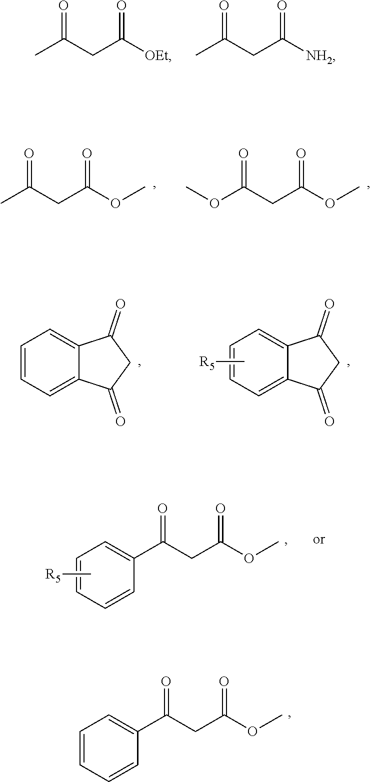
wherein R₅ or R₆ is cyano, carboxy, or halo.

25. (canceled)

26. (canceled)

27. The polynucleotide of claim 19, wherein the exocyclic amine of the 5-caC participates in the product rearranging.

28. (canceled)

29. The polynucleotide of claim 19, wherein the product comprises: 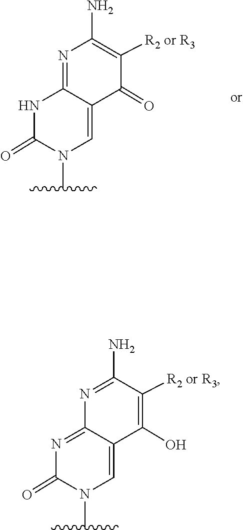
wherein R₂ or R₃ includes an electron withdrawing group.

30. A double-stranded polynucleotide, comprising:
the polynucleotide of claim 19; and
a second polynucleotide hybridized to the polynucleotide and comprising adenine (A) at a location complementary to the product.
